(19) United States
(12) Patent Application Publication
(10) Pub. No.: US 2020/0277568 A1
(43) Pub. Date: Sep. 3, 2020

(54) Small Molecule-Induced Method for Directly Reprogramming Human Fibroblasts into Liver Cells

(71) Applicant: Transcend Cytotherapy Co.,Ltd, Jiangsu (CN)

(72) Inventor: Peilin Zhang, Jiangsu (CN)

(21) Appl. No.: 16/645,819

(22) PCT Filed: Sep. 17, 2018
(86) PCT No.: PCT/CN2018/105936

§ 371 Date: Apr. 13, 2020

(30) Foreign Application Priority Data
Sep. 15, 2017 (CN) 201710833549.2

(51) Int. Cl. C12N 5/071

(52) U.S. Cl. CPC C12N 5/067; C12N 2506/1307; C12N 2501/72; C12N 2501/15

(57) ABSTRACT

This disclosure relates to a method for direct reprogramming (transdifferentiation) of human fibroblasts into hepatocytes by small molecules. A method and a small molecule composition for direct reprogramming (transdifferentiation) of human fibroblasts into hepatocytes based on a mechanism of chemically induced cell direct reprogramming are disclosed. The small molecule composition can be developed into drug or prodrugs for the treatment of hepatic fibrosis (Cirrhosis), pulmonary fibrosis and fibrosis diseases of other organ or tissue in human. This disclosure also discloses a cell transdifferentiation medium and a reagent prepared by the small molecule composition.

### TECHNICAL FIELD

This disclosure belongs to the cross-disciplinary fields of cell biology, stem cell biology (cell reprogramming), medicine and pharmacy; more specifically, the present disclosure relates to a method for directly reprogramming (transdifferentiating) human fibroblasts into hepatocytes by small molecules.

### BACKGROUND TECHNIQUE

China is a country with high incidence of liver diseases. There are many patients with hepatitis A, hepatitis B, fatty liver, alcoholic liver, cirrhosis (hepatic fibrosis), etc. Among them, acute and chronic hepatic failure caused by various liver diseases, chemicals, trauma and other reasons can quickly develop into critical conditions with high mortality rates. Liver transplantation is an effective way to treat end-stage liver diseases, especially liver failures. However, donor organ shortage has led many patients, especially those with acute hepatic failure, to lose their opportunities for treatment. Hepatocyte transplantation, bioartificial liver, and transplantation of bioengineered liver are important alternatives to liver transplantation, and have thus been widely studied. How to obtain sufficient quantity of human hepatocytes suitable for clinical use has become the focus of international researches. Till now, the main ways to obtain functional hepatocytes are as follows: 1) Isolation of primary hepatocytes from the donor liver; 2) Directional differentiation of stem cells or induced pluripotent stem cells into hepatocytes; 3) Transdifferentiation of somatic cells such as fibroblasts into hepatocytes. Application of donor liver-isolated primary hepatocytes is limited due to severe shortage of donor organs. Stem cell differentiation, in the meantime, has likely become the most practical method for obtaining sufficient quantity of functional hepatocytes, due to the infinite proliferation potential of stem cells. However, current directional differentiation methods have one or more of the following shortcomings: low differentiation efficiency, certain functions missing in differentiated hepatocytes, possibility of immune rejection, potential carcinogenic risk caused by incomplete stem cell differentiation and high differentiation cost, etc. As a result, stem cell differentiated hepatocytes do not meet the requirements for clinical application. Direct reprogramming (transdifferentiation) of fibroblasts into hepatocytes has attracted immense interests in recent years because it avoids the potential risks of stem cells by bypassing the iPS stage (Induced pluripotent stem cell) in the reprogramming process. However, current methods for transdifferentiating fibroblasts into hepatocytes requires introduction of exogenous genes, which poses a risk of carcinogenesis and makes the cells unsuitable for clinical application. Another method involves using small molecules to reprogram gastric epithelial cells into endoderm stem cells (pluripotent reprogramming), which are then differentiated into hepatocytes, islet cells, and small intestinal epithelial cells. Application of this method also faces many challenges, as in vivo pluripotent reprogramming is not only difficult but introduces carcinogenic risks; in addition, the second step of in vivo directional differentiation of stem cells is also difficult and risky to complete, making the method unpractical for clinical use. Even in vitro applications of the above-mentioned methods also have various deficiencies such as complex process, increased experimental steps, difficulty in quality control, high costs and clinical application risks. Therefore, how to avoid the introduction of exogenous factors (genes), and use only small molecules to directly reprogram (transdifferentiate) fibroblasts into hepatocytes, while overcoming the deficiencies of the above-mentioned transdifferentiation or directional differentiation methods, are bottleneck problems that must be solved in order to increase clinical application of hepatocyte transplantation.

Cell reprogramming refers to the conversion of cells from one type to another, which is a process of cell fate alteration through modulating cell signaling pathways and epigenetic modifications. The so-called “epigenetics” in biology means heritable phenotype changes that do not involve alterations in the DNA sequence. Cell reprogramming can be achieved through nuclear transfer (NT), cytoplasmic incubation, cell fusion, and transcription or chemical factor-induced cell reprogramming. Among them, induced cell reprogramming is currently recognized as the research field with most breakthroughs (Note: The cell reprogramming mentioned in this method refers to transcription or chemical factor-induced cell reprogramming). Induced cell reprogramming includes: (1) pluripotent reprogramming by reversing differentiated cells to pluripotent or totipotent state; (2) Direct reprogramming by direct transforming one type of differentiated cell into another without going through the pluripotent stem cell stage (also known as transdifferentiation/lineage reprogramming). Cell reprogramming induced by small molecules only is also known as chemical-induced cell reprogramming. Induction by small molecules is considered to be one of the most promising methods for improving the reprogramming process. Small molecules can partially or even completely replace transcription factors to achieve cell reprogramming. They can also eliminate many of the disadvantages of introducing exogenous genes (transcription factors), as they can more easily penetrate cells, lower the chance of causing genetic mutations, simplify the experiment steps, reduce the risk of carcinogenesis, shorten the reprogramming process, and improve reprogramming efficiency. Especially from the perspective of clinical application, it is highly important that the direct reprogramming (hereinafter referred to as: transdifferentiation) induction factors can be developed into pharmaceutical drugs, and can complete in-situ transdifferentiation of cells in vivo. For example, when damages or diseases occur to important organs such as the heart, brain and liver, fibroblasts or even diseased cells can be transdifferentiated in-situ into corresponding normal organ cells to cure the injury or disease. Breakthroughs have been made in the field of chemical-induced cell reprogramming in recent years. Not only can fibroblasts now be reprogrammed into iPS cells, but they can also be transdifferentiated into neural cells and cardiomyocytes. However, transdifferentiation of human fibroblasts into hepatocytes using only small molecules for induction has not been reported.

### SUMMARY OF THIS DISCLOSURE

The purpose of the present disclosure is to provide a method for directly reprogram (transdifferentiate) human fibroblasts into hepatocytes through induction of small molecule compositions and the small molecule compositions thereof; the small molecule composition can be combined with drug carrier or an excipient in order to be developed into drug or prodrug for clinical treatment of hepatic fibrosis (cirrhosis), pulmonary fibrosis and fibrosis disease of other organs or tissues. By adding aqueous or organic solvents, basic medium or serum-free medium, it can also be prepared into cell transdifferentiation reagents or media, or be used to transdifferentiate human fibroblasts into hepatocytes, and provide a source of hepatocytes for scientific research, medical or clinical applications such as hepatocyte transplantation for the treatment of hepatic failure.

In a first aspect of the present disclosure, it provided a small molecule composition (or formula) for chemically inducing human fibroblasts to directly reprogram (transdifferentiate) into hepatocytes, the composition comprising a GSK3β inhibitor, a TGF β inhibitor and a G9a histone methyltransferase (G9aHMTase) inhibitor; or the composition consists of a GSK3β inhibitor, a TGFβ inhibitor, and a G9aHMTase inhibitor.

In a preferred example, the composition comprises:

GSK3β inhibitor: 5-80 parts by weight;

TGFβ inhibitor: 0.1-50 parts by weight; and

G9aHMTase inhibitor: 0.1-50 parts by weight.

In another preferred example, the composition may be a composition in solution state, comprising:

GSK3β inhibitor: final concentration: 0.1-20 uM; and

TGFβ inhibitor: final concentration: 0.01-20 uM; and

G9aHMTase inhibitor: final concentration: 0.01-20 uM.

In another preferred example, the composition comprises:

GSK3β inhibitor: 10-70 parts by weight; and

TGFβ inhibitor: 0.5-40 parts by weight; and

G9aHMTase inhibitor: 0.5-40 parts by weight.

In another preferred example, the composition may be a composition in solution state, including:

GSK3β inhibitor: final concentration: 0.5-10 uM; and

TGFβ inhibitor: final concentration: 0.05-10 uM; and

G9aHMTase inhibitor: final concentration: 0.05-10 uM.

In another preferred example, the combined weight of GSK3β inhibitor, TGFβ inhibitor, and G9aHMTase inhibitor adds up to 0.01 to 99.9% of the total weight of the composition; more preferably 50 to 99.9%; 0.1 to 50% in the solution state, such as 1%, 5%, 10%, 20%, 30%, etc.

In another preferred example, in the composition, the GSK3 β inhibitor (such as GSK3 β inhibitor CHIR99021), the TGF β inhibitor (such as TGF β inhibitor SB431542 or/and A83-01), the G9aHMTase inhibitor (such as BIX01294) exist in parts by weight: (5-80):(0.1-50):(0.1-50); preferably, (10-70):(0.5-40):(0.5-40); or in solution state at a molar concentration ratio: (0.1-20):(0.01-20):(0.01-20); preferably, (0.5-10):(0.05-10):(0.05-10).

The weight unit of the above weight parts (parts by weight) can be any weight unit such as kilogram (kg), milligram (mg), microgram (ug); the molar unit of the molar concentration can be: Molar (M), Millimolar (mM), Micromolar (uM) and any other molar concentration unit.

In addition, when the composition is applied to large animals and patients, the effective dosage of large animals or humans (including solid or solution dosage conversion) is converted by the corresponding professional conversion formula according to the dosage of small animals, which also belongs to the protection scope of the present disclosure.

In another preferred example, the GSK3β inhibitor includes: same type of GSK3β signaling pathway inhibitors or compounds that have the same function or engage the same target, as represented by CHIR-99021, BIO, IM-12, TWS119, 1-Azakenpaullone, CHIR-98014, Tideglusib, AR-A014418, LY2090314, SB216763, AZD1080, etc., or the equivalent pharmaceutical products, analogs, isomers, and/or a salt, hydrate or precursor thereof, or a combination thereof; preferably, the GSK3β inhibitor is CHIR-99021, BIO or TWS119;

The TGFβ inhibitor includes: same type of TGFβ signaling pathway inhibitors or compounds that have the same function or engage the same target, as represented by SB431542, A83-01, SB525334, LY2109761, RepSox, SD-208, GW788388, SB505124, EW-7197, Galunisertib, etc., or the equivalent pharmaceutical products, analogs, isomers, and/or a salt, hydrate or precursor thereof, or a combination thereof; preferably, the TGFβ inhibitor is SB431542, A83-01 or LY2109761;

The G9aHMTase inhibitors include: same type of G9HMTase inhibitors or compounds that have the same function or engage the same target, as represented by: BIX01294, UNC0638, A-366, UNC0631, BRD4770, UNC0224, UNC0646, UNC0642, etc.; or the equivalent pharmaceutical products, analogs, isomers, and/or a salt, hydrate or precursor thereof, or a combination thereof; preferably, the GIXaHMTase inhibitor is BIX01294 (or BIX-01294) or UNC0638.

In another preferred example, the composition is a drug or a pharmaceutical composition for treating hepatic fibrosis (cirrhosis), pulmonary fibrosis, and fibrosis of other organs or tissues, and further comprises a pharmaceutically acceptable carrier or excipient agents, wherein the carriers or excipients include, but are not limited to, one or more selected from the group consisting of:

Water, saline, phosphate buffer or other aqueous solvents;

DMSO (dimethyl sulfoxide), glycerol and ethanol or other organic solvents;

Microspheres, liposomes, microemulsions or macromolecular surfactants;

Colloidal drug-loading system or macromolecular drug-loading system; or

Preservatives, antioxidants, flavoring agents, fragrances, solubilizers, emulsifiers, pH buffering substances, adhesives, fillers, lubricants or other pharmaceutical excipients.

In another preferred example, the pharmaceutical dosage form that the composition can be prepared into includes (but is not limited to):

Solid dosage forms, including (but are not limited to) powders, pulvis, tablets, pills, capsules, sustained-release agents, controlled-release agents;

Liquid dosage forms, including (but are not limited to) injections, infusions, suspensions, or other liquid dosage forms;

Gas dosage form; or

Semi-solid dosage form.

In another preferred example, an aqueous solvent or an organic solvent can be added to the composition to prepare a reagent for inducing transdifferentiation of fibroblasts into hepatocytes for scientific research; a basic cell culture medium or a serum-free medium can be added to prepare culture medium for the same purpose. Each component of the composition exists in a basal cell culture medium containing 5-20% calf serum, 1% penicillin mixed solution (100×) or a serum-free medium containing various cytokines or growth factors, but the composition does not comprise basal cell culture medium or serum-free medium.

In another preferred example, the composition can be used to transdifferentiate human fibroblasts into hepatocytes, and provide a source of hepatocytes for scientific research, medical applications, and clinical applications such as hepatocyte transplantation.

In another aspect of the present disclosure, the composition is used for the development or preparation of a drug or prodrug (or drug formula) for treating hepatic fibrosis (cirrhosis), pulmonary fibrosis and fibrosis diseases of other organ or tissue; or for preparing a culture medium or reagent for inducing human fibroblasts to transdifferentiate into hepatocytes; or for transdifferentiating human fibroblasts into hepatocytes to provide a source of hepatocytes for scientific research, medical applications and clinical applications such as hepatocyte transplantation.

In another aspect of the present disclosure, a method for inducing human fibroblasts to directly reprogram (transdifferentiate) into hepatocytes is provided, characterized by that the method involves applying any of the foregoing compositions to induce and regulate the transdifferentiation of human fibroblasts into hepatocytes.

In another preferred embodiment, a method for preparing a medium or reagent for inducing human fibroblasts to directly reprogram (transdifferentiate) into hepatocytes and the experiment step thereof are provided, comprising:

(1) Formulation of concentrated liquid reagent: each component of the foregoing compositions is dissolved in an organic solvent or an aqueous solvent to prepare a concentrated liquid reagent; preferably, the organic solvent includes dimethyl sulfoxide, and the aqueous solvent includes: water, physiological saline, phosphate buffer solution;

(2) Culture medium for transdifferentiation of fibroblast into hepatocyte: Dilute the concentrated solution reagent in step (1) into a basal cell culture medium containing 5-20% calf serum or a serum-free medium comprising a variety of cytokines or growth factors (so that the concentration of each component conforms to the final concentration defined in any of the foregoing compositions), to obtain a medium for inducing fibroblasts to transdifferentiate into hepatocytes; wherein, the content in percentage of each component in the medium can also be increased or decreased by 50%, preferably by 30%, more preferably by less than 20%, such as 10% and 5%;

(3) Inducing fibroblasts to transdifferentiate into hepatocytes: suspend fibroblasts in a basal cell culture medium comprising 5-20% calf serum or a serum-free medium comprising various cytokines or growth factors and plate them, after the cells have adhered, replace basal medium with the transdifferentiation medium of step (2), culture at 37° C., and change the medium every 2-4 days; the cells are passaged every 3-15 days.

(4) Passaging of hepatocytes derived from fibroblasts transdifferentiation: Discard the original culture solution, wash the cells once with PBS, add cell digestion solution to digest the cells at 37° C. for 1-5 min, then stop cell digestion, centrifuge and discard the supernatant. Resuspend cell pellets and plate them at 1:1-1:3 passage. Cells should be cultured according to the method above using the transdifferentiation medium of step (2), and solution should be changed every 2-4 days. The digestive solution used includes trypsin, EDTA, Acutase, TrypleE, etc. Passage cells every 3-15 days.

(5) Harvesting the hepatocytes derived from fibroblasts transdifferentiation: After culturing and passaging the cells according to experiment steps (3) and (4) mentioned above for 2-4 weeks. Fibroblasts can complete hepatic transdifferentiation, and transdifferentiated hepatocytes can be obtained.

In another aspect of the present disclosure, a kit/test kit for inducing direct reprogramming (transdifferentiation) of human fibroblasts into hepatocytes is provided. The kit/test kit comprises: any of the composition described above; or a drug or a pharmaceutical formula developed based on the composition for treating hepatic fibrosis (cirrhosis), pulmonary fibrosis, and other organ or tissue fibrosis; or a reagent or culture prepared based on the composition for scientific research.

According to any of the foregoing aspects, the human fibroblasts include, but are not limited to, fibroblasts from different tissues or organs of the human body, such as skin fibroblasts, hepatic fibroblasts (hepatic stellate cells, HSCs)), lung fibroblasts, kidney fibroblasts, pancreatic fibroblasts, and fibroblasts from other tissues or organs in the human body. Preferred are human skin fibroblasts and hepatic fibroblasts (hepatic stellate cells).

Other aspects of this disclosure will be apparent to those skilled in the art from the disclosure herein.

### DESCRIPTION OF THE DRAWINGS

FIG. 1. Morphological comparison of human fibroblasts (HFF) with fibroblast-transdifferentiated hepatocyte-like cells (ciHep). Left: human fibroblasts (HFF); right: human fibroblasts-transdifferentiated hepatocyte-like cells (ciHep). The results show that after transdifferentiation of human fibroblasts, their morphology changes significantly, which is consistent with the morphology of hepatocytes.

FIG. 2. Flow cytometric analysis of liver-specific marker staining in fibroblast-transdifferentiated hepatocyte-like cells. This result indicates that the percentage of cells with positive staining for hepatocyte-specific markers in the transdifferentiated hepatocyte-like cells is very high, while the embryonic hepatocyte marker AFP has almost no expression. This proves that the transdifferentiation medium and the culture method of the present disclosure can transdifferentiate human fibroblasts into mature, functional human hepatocytes.

FIG. 3. Comparison of albumin production by fibroblast-transdifferentiated hepatocyte-like cells and primary hepatocytes. Among them, PHH is: primary hepatocytes; 1-4 are: human fibroblast-transdifferentiated hepatocyte-like cells. The results show that the transdifferentiated hepatocyte-like cells have the capability of albumin production, which is unique to human hepatocytes.

FIG. 4. Urea production by human fibroblast-transdifferentiated hepatocyte-like cells. The source of urea is ammonia in the blood, and the amount of urea produced reflects the detoxifying ability of hepatocytes. This result shows that the hepatocyte-like cells derived from human fibroblast transdifferentiation have the function of urea production, which is unique to hepatocytes.

FIG. 5. Glycogen staining of human fibroblast-transdifferentiated hepatocyte-like cells. The degree of staining represents the glycogen storage ability of hepatocytes. Above panel: Glycogen staining of human fibroblast-transdifferentiated hepatocyte-like cells. Bottom panel: human fibroblasts. Fibroblast-transdifferentiated hepatocyte-like cells show positive staining for glycogen, while human fibroblasts show negative staining, which proves that hepatocyte-like cells obtained by the method of the present disclosure have the same glycogen storage ability as human hepatocytes.

FIG. 6. Induction of P450 enzyme (CYP3A4 and CYP1A2) activity in fibroblast-transdifferentiated hepatocyte-like cells. This result shows that the transdifferentiated hepatocyte-like cells obtained by the method of the present disclosure have P450 metabolic enzyme activity unique to hepatocytes. The y-axis in the figure is the relative activity of the enzyme.

FIG. 7. Comparison of fat staining of human fibroblasts and fibroblast-transdifferentiated hepatocyte-like cells. Positive staining indicate the ability of the transdifferentiated hepatocytes to metabolize fat. Left: human fibroblasts; right: human fibroblasts-transdifferentiated hepatocyte-like cells. The results show that the transdifferentiated hepatocytes have positive staining for fat, and the fibroblasts have negative staining; which proves that the hepatocyte-like cells obtained by the transdifferentiation method have the hepatic ability to metabolize fat.

FIG. 8. Fat uptake of human fibroblasts transdifferentiated hepatocytes. The degree of staining demonstrates the ability of hepatocytes to take up fat. The results show that the hepatocyte-like cells obtained through transdifferentiation method in present disclosure has the function of fat uptake and storage of hepatocytes, while fibroblasts do not have this function.

FIG. 9. ICG uptake of human fibroblast-transdifferentiated hepatocyte-like cells. The degree of staining demonstrates the ability of hepatocytes to take up and excrete foreign entities. The results show that fibroblast-transdifferentiated hepatocyte-like cells have the ability of human hepatocytes to take up and excrete foreign entities, which is a function unique to hepatocytes.

FIG. 10. Comparison of morphology and functions (glycogen staining, oil red staining) between hepatocyte-like cells (Lx2-ciHep) derived from transdifferentiation of human hepatic stellate cells (HSC) with control group hepatic stellate cells. Human hepatic stellate cells (HSCs) obtain the morphological and functional characteristics of hepatocytes after transdifferentiation.

FIG. 11. Experiment results of using the aforementioned composition-based reagent to treat mice model with hepatic fibrosis through oral administration. The results show that transdifferentiation of hepatic fibroblasts induced by the small molecule composition led to significantly reduced or alleviated hepatic fibrosis in animals (Sirius red staining is significantly reduced), thus the composition achieves therapeutic effect on hepatic fibrosis (cirrhosis) disease.

FIG. 12. Human lung fibroblasts (control) are transdifferentiated into hepatocyte-like cells (treated). The transdifferentiated cells have hepatocyte morphology. The results of this experiment show that human lung fibroblasts obtain hepatocyte morphology after transdifferentiation.

FIG. 13. Human lung fibroblasts (control) express hepatocyte-related genes after transdifferentiation into hepatocyte-like cells (treated). The results of this experiment show that lung fibroblast-transdifferentiated cells highly express hepatocyte-related genes. This indicates that human lung fibroblasts have been transdifferentiated into hepatocyte-like cells.

### DETAILED DESCRIPTION

After intensive research, the present inventors have disclosed a method for inducing human fibroblasts to directly reprogram (transdifferentiate) into hepatocytes using a small molecule composition and the small molecule composition thereof. A drug carrier or excipients can be added to the small molecule composition to develop drugs or prodrugs for clinical treatment of hepatic fibrosis (cirrhosis), pulmonary fibrosis and fibrosis of other organ or tissue. Aqueous or organic solvents, basal or serum-free media can be added to prepare reagents or culture media for scientific research. The composition can be used to transdifferentiate human fibroblasts into hepatocytes, and provide a source of hepatocytes for scientific research applications, medical applications and clinical applications such as hepatocyte transplantation. The method for chemically inducing human fibroblasts to directly reprogram (transdifferentiate) into human hepatocytes by small molecule composition can be applied to different types of human fibroblasts such as skin fibroblasts, hepatic fibroblasts (hepatic stellate cells), lung fibroblasts and fibroblasts of other tissues and organs, etc. The hepatocyte-like cells obtained through transdifferentiation have normal hepatocyte functions.

Basic Mechanism

Induced cell reprogramming includes: (1) pluripotent reprogramming by reversing differentiated cells back to pluripotent or totipotent states; (2) Direct reprogramming by converting one differentiated cell type to another without passing through the pluripotent stem cell stage (transdifferentiation/lineage reprogramming). Cell reprogramming induced by small molecules only is also called chemically induced cell reprogramming.

Chemically induced cell direct reprogramming (transdifferentiation) refers to the process of changing cell fate by regulating cell signal pathways and epigenetics without modifying gene sequences.

With the development of stem cell science, cell reprogramming has evolved from using introduction of exogenous transcription factors (genes) to using small molecule combinations that regulate cell signaling pathways and epigenetics to achieve “chemically induced pluripotent cell (iPSC) reprogramming” (Hongkui Deng et al., Science. 341, 651-4, 2013) and “chemically induced cell direct reprogramming” (Li X et al., Cell Stem Cell; 17 (2): 195-203, 2015; Hu W et al., Cell Stem Cell. 17 (2): 204-212, 2015). In addition, the starting and target cell types have also been expanded from reprogramming of differentiated cell into pluripotent stem cell to direct reprogramming of one differentiated cell into another differentiated cell.

Based on previous reports, multiple types of normally differentiated cells can be reprogrammed into one type of cell (see Table 1). It can thus be concluded that the key to reprogramming is not the starting cell type, but rather the correct combination of induction factors that can construct and maintain the gene expression profile or biological behavior of the target cell, as well as break various “energy barriers” during the cell reprogramming process.

Table 1 Examples of multiple starting cells reprogrammed to the same target cell

| | Cell reprogramming | |
|---|---|---|
| Starting cells | type | Target Cell |
| Fibroblasts, hepatocytes, | Inducing pluripotent | Induced pluripotent |
| Neural stem cells, | cell reprogramming | cells (iPSCs) |
| foreskin cells, | | |
| Keratinocytes, Amniotic | | |
| fluid cells, blood cells, | | |
| endothelial cells, etc. | | |
| Fibroblast, Astroglial | Inducing cell direct | Neurons |
| cell, hepatocytes, | reprogramming | |
| Perivascular cells | (transdifferentiation) | |

Therefore, the inventor believes that as long as the induction factors or compounds that can break various “energy barriers” and construct and maintain the gene expression profile unique to the target cell are found, various differentiated cells of different germ layers, including fibroblasts, may change their cell fate via cell reprogramming. Therefore, the present method screens and designs the small molecule combinations according to the above-mentioned mechanism, to induce and regulate fibroblasts to transdifferentiate into hepatocytes (target cells).

This disclosure first selects easily obtainable skin fibroblasts as the breakthrough point, then screens for small molecule combination that can construct and maintain the unique gene expression profile of normal hepatocytes (target cells), and can break “energy barriers” in the reprogramming process, thus constructing a method for direct reprogramming (transdifferentiation) of fibroblasts into hepatocytes. This innovative method has the following advantages: \textcircled{1} small molecules are stable in nature; in addition, the time of action, dose and combination can be easily manipulated, the effect they achieve is also consistent and reliable; \textcircled{2} hepatocytes derived from transdifferentiation of fibroblasts have morphology and functions of normal mature human hepatocyte; \textcircled{3} the fibroblasts in this method can be obtained from the patient and transdifferentiate into autologous hepatocytes, which have two major advantages: first of all, they can be applied clinically as they can minimize or avoid immune rejection risk in hepatocyte transplantation; secondly, they can be used for building crowd-representative hepatocyte bank for liver toxicity and efficacy screening of new drugs; \textcircled{4} this method avoids the introduction of exogenous transcription factors or genetic manipulation, which can cause carcinogenic risks, thus making it safer and more reliable; \textcircled{5} This method uses chemical-induced direct reprogramming, and bypasses the stage of induced pluripotent stem cells (iPSC), thus avoiding the risk of stem cell-related carcinogenesis; \textcircled{6} small molecules can be more easily developed into pharmaceutical drug; and since this method can induce in situ cell transdifferentiation in vivo, the composition used can be developed or prepared into drugs or prodrugs for treating human hepatic fibrosis (cirrhosis), pulmonary fibrosis, and fibrosis diseases of other organ or tissue.

The known mechanism of the present disclosure mainly relies on the combination of three types of small molecules, including GSK3β inhibitor, TGFβ inhibitor, and G9aHMTase inhibitor, which, through multi-target induction, modulate signal pathways, epigenetics, and chemical biology of cells to transdifferentiate fibroblasts into hepatocytes. Hepatic fibrosis can be significantly reduced or alleviated through in situ transdifferentiation of fibroblasts in vivo and the regulation of the internal environment by the small molecule composition. Furthermore, hepatocyte function can be improved or restored to help achieving the goal of treating hepatic fibrosis (cCirrhosis) and related hepatic failure. Through the same mechanism, the small molecule composition can reduce or alleviate fibrosis of other organs or tissues through in situ transdifferentiation of lung fibroblasts, renal fibroblasts, and fibroblasts in other organs or tissues, thus achieving therapeutic effects on fibrotic diseases. It should be noted that GSK3β inhibitor, TGFβ inhibitor, and G9aHMTase inhibitor in the composition include three series of small molecules of the same type, function, or engagement target with similar effect and result. Different combinations formed by different molecules in the series can all induce fibroblasts to transdifferentiate into hepatocytes to different extent. Therefore, the same type of small molecule compounds with the same function or the same engagement target, or with the same effect on the same signal pathway, and their combination that can induce and regulate the human fibroblasts to transdifferentiate into hepatocytes all fall into the scope of protection of the present disclosure.

On the other hand, human fibroblasts, also known as fibroblasts, are the main cellular component of loose connective tissue and are differentiated from mesenchymal cells at the embryonic stage. Fibroblasts can be divided into fibroblasts and fibrocytes according to different functional activity states. Fibroblasts have strong functional activities, cytoplasm is weakly alkaline, and has obvious protein synthesis and secretion activities. When they reach mature or static stage, fibroblasts are called fibrocytes, but under certain conditions, the two can transform into each other. Fibroblasts have different types and are present in various tissues or organs in the body. They have different names and characteristics in different tissues or organs, including skin fibroblasts, hepatic fibroblasts (hepatic stellate cells), lung fibroblasts, pancreatic fibroblasts, etc. The human fibroblasts described in this method include, but are not limited to, fibroblasts from different tissues or organs of the human body, such as skin fibroblasts, hepatic fibroblasts (hepatic stellate cells), lung fibroblasts, renal fibroblasts, pancreatic fibroblasts, and fibroblasts from other tissues or organs in the body. Preferred are human skin fibroblasts and hepatic fibroblasts (hepatic stellate cells).

The human fibroblasts according to the method of the present disclosure include, but are not limited to, fibroblasts from different tissues or organs of the human body, such as skin fibroblasts, hepatic fibroblasts (hepatic stellate cells), lung fibroblasts, renal fibroblasts, pancreatic fibroblasts, and fibroblasts from other tissues or organs in the body. Preferred are human skin fibroblasts and hepatic fibroblasts (hepatic stellate cells).

Pharmaceutical Composition and Application Thereof

After extensive research, the inventors proposed for the first time that simultaneously inhibiting GSK3β signaling pathway, TGFβ signaling pathway and G9aHMTase to varying degrees, can induce fibroblasts to transdifferentiate into hepatocytes.

The small molecule composition has the potential to be developed into a new drug or prodrug for the treatment of hepatic fibrosis (cirrhosis), pulmonary fibrosis, renal fibrosis and fibrosis diseases of other organ or tissue; and it can be directly prepared as medium or reagent for scientific use for chemically inducing fibroblasts to directly reprogram (transdifferentiated) into hepatocytes; it can also be used to transdifferentiate human fibroblasts into hepatocytes, thus providing a source of hepatocytes for scientific research applications, medical and clinical applications such as hepatocyte transplantation for hepatic failure.

It should be understood that, in addition to the specific GSK3β inhibitors and TGFβ inhibitors listed in the examples of the present disclosure, those inhibitor of the same type that can inhibit the GSK3β signaling pathway and TGFβ signaling pathway, modulate the same target, have the same function, or can produce the same effect and results, can also achieve the same technical effect and should be included in the present disclosure.

Similarly, G9aHMTase inhibitors other than the G9aHMTase inhibitors listed in the examples of the present disclosure that have the same function or can modulate the same target, can also achieve the same technical effect, which should also be included in the present disclosure.

As used herein, the terms “comprising” or “including” include “including,” “consisting essentially of,” and “consisting of.”

As used herein, the term “consisting essentially of” means that in addition to the essential ingredients or essential ingredients, small amount of ingredients of less importance and/or impurities that do not affect the active ingredients may also be contained in the composition. For example, sweeteners to improve taste, antioxidants to prevent oxidation, and other pharmaceutical additives, carriers, and excipients commonly used in the art can also be included.

As used herein, the term “pharmaceutically acceptable” ingredients are substances that can be applied to humans and/or animals without excessive adverse side effects (such as toxicity, irritation, and allergies), that is, substances that have a reasonable benefit/risk ratio; Such as pharmaceutical carriers or excipients commonly used in the art.

As used herein, the term “effective amount” refers to an amount that is functional or active in humans and/or animals and acceptable for humans and/or animals.

As used herein, the term “pharmaceutically acceptable carrier or excipient”, wherein the carrier refers to a system that can alter the way a drug enters the body and its distribution in the body, control the rate of drug release, and deliver the drug to a targeted organ; the pharmaceutical carrier itself is not an essential active ingredient and is not excessively toxic after administration. Suitable carriers are well known to those of ordinary skill in the art and include, but are not limited to: water, saline, phosphate buffer, and other aqueous solvents; DMSO (dimethyl sulfoxide), glycerol and ethanol, and other organic solvents; microspheres, liposomes, microemulsions, macromolecular surfactants; colloid-type drug-loading systems, new macromolecular drug-loading systems, new drug carriers, and other pharmaceutical carriers; wherein the excipients refer to additives in the pharmaceutical preparations other than the main drug, and can also be called supplements. For example, the binders, fillers, disintegrating agents, lubricants in tablets; wine, vinegar, medicinal fluid in Chinese medicine pills; matrix parts in semi-solid preparations such as ointments, creams; preservatives antioxidants, flavoring agents, fragrances, solubilizers, emulsifiers, solubilizers, osmotics, colorants, etc. in liquid preparations can all be called excipients.

The general requirements for excipients are stable in nature, no contraindication to the main drug, no side effects, no effect on the efficacy, and no deformation, dry cracking, mildew, moth-eaten under normal temperature, harmless to the human body, no physiological effects, no chemical or physical interactions with the main drug, and no influence on the dosage of the main drug. A full discussion of pharmaceutically acceptable carriers or excipients can be found in Remington's Pharmaceutical Sciences (Mack Pub. Co., N.J. 1991). Carriers or excipients include, but are not limited to: water, saline, phosphate buffer and other aqueous solutions; organic solvents such as DMSO (dimethyl sulfoxide), glycerol, and ethanol; microspheres, liposomes, microemulsions, macromolecular surfactant; colloidal drug loading systems, new macromolecular drug loading systems, new drug carriers, and other pharmaceutical carriers; preservatives, antioxidants, flavoring agents, fragrances, co-solvents, emulsifiers, pH buffer substances in liquid preparations; binders, fillers, lubricants and other pharmaceutical excipients in tablets.

As used herein, a pharmaceutical dosage form in the term “pharmaceutical dosage form that the composition can be prepared into” refers to: a pharmaceutical application form prepared to meet the needs of treatment or prevention, called a pharmaceutical dosage form; the pharmaceutical dosage form that can be prepared by any of the compositions of the present disclosure includes but not limited to: powders, pulvis, tablets, pills, capsules, sustained release dosages, controlled release dosages and other solid dosage forms; injections, infusions, suspensions and other liquid dosage forms, as well as gas dosage forms, semi-solid dosage forms and other dosage forms.

As used herein, “part(s) by weight” or “portion(s) by weight” are used interchangeably, and the parts by weight may be any fixed weight expressed in micrograms, milligrams, grams, or kilograms (such as 1 ug, 1 mg, 1 g, 2 g, 5 g, or kg, etc.). For example, a composition composed of 1 part by weight of component a and 9 parts by weight of component b may be 1 g of component a+9 g of component b, or 10 g of component a+90 g of component b. In the composition, the percentage content of a certain component=(parts by weight of the component/sum of parts of weight of all components)×100%. Therefore, in a composition composed of 1 part by weight of component a and 9 parts by weight of component b, the content of component a is 10% and component b is 90%.

In addition, in the solution state, the “parts by weight” may be converted into “moles”; the “parts by weight” may also be converted into “molar concentration ratios”. The weight unit of the weight part ratio may be: any weight unit such as kilogram (kg), milligram (mg), microgram (ug); the molar unit of the molar concentration ratio may be: Molar (M), Millimolar (mM), Micromolar (uM) and any other molar concentration units;

GSK3 β inhibitor (such as GSK3 β inhibitor CHIR99021), TGF β inhibitor (such as TGF β inhibitor SB431542 or/and A83-01), G9aHMTase inhibitor (such as G9aHMTase inhibitor BIX01294), present in parts by weight: (5-80):(0.1-50):(0.1-50); preferably, (10-70):(0.5-40):(0.5-40); or in a solution state present at a molar concentration ratio: (0.1-20):(0.01-20):(0.01-20); preferably, (0.5-10):(0.05-10):(0.05-10).

As a preferred embodiment of the present disclosure, the components and weight part ratios included in the composition are shown in Table 2 or the molar concentration ratios are shown in Table 3 (solution state).

TABLE 2

| | | Preferred |
|---|---|---|
| | Weight part ratio | weight part |
| Components | (weight unit: kg, mg, ug . . .) | ratio |
| GSK3β | 5-80, for example 5, 10, 15, 20, 40, 50, 60, | 10-70 |
| inhibitor | 70, 80 | |
| TGFβ | 0.1-50, for example 0.1, 0.5, 1, 3, 5, 10, 20, | 0.5-40 |
| inhibitor | 40, 50 | |
| G9aHMTase | 0.1-50, for example 0.1, 0.5, 1, 3, 5, 10, 20, | 0.5-40 |
| inhibitor | 40, 50 | |

TABLE 3

| | | Preferred mole |
|---|---|---|
| | Mole concentration ratio | concentration |
| Components | (mole unit: M, mM, uM . . .) | ratio |
| GSK3β | 0.1-20, for example 0.1, 0.5, 1, 3, 5, 10, | 0.5-10 |
| inhibitor | 15, 20 | |
| TGFβ | 0.01-20, for example 0.01, 0.5, 1, 3, 5, 10, | 0.05-10 |
| inhibitor | 15, 20 | |
| G9aHMTase | 0.01-20, for example 0.01, 0.5, 1, 3, 5, 10, | 0.05-10 |
| inhibitor | 15, 20 | |

The range of formulations in Tables 2 and 3 can be used as a reference guide. It should be understood, however, that when used in the development and manufacturing of pharmaceutical drugs, the effective dose of the composition used may vary with the mode of administration and the body condition of a patient with hepatic fibrosis (cirrhosis), pulmonary fibrosis, and fibrosis diseases of other organ or tissue to be treated, or disease severity. In addition, when used in vivo, “weight/kg (body weight)” is usually used as a dosage unit. When the small molecule composition is applied to large animals and patients with liver disease, the small animal composition is converted by the corresponding professional conversion formula and converted into the effective dosage of large animals or humans (including solid or solution dosage conversion) also falls within the protection scope of the present disclosure.

As used in the present disclosure, the “GSK3β inhibitor” refers to a general term of an inhibitor capable of inhibiting the GSK3β signaling pathway in a cell, including but not limited to: CHIR-99021, BIO, IM-12, TWS119 and other GSK3β signaling pathway inhibitors or compounds of the same type, have the same function or engage the same target:

CHIR-99021 (CT99021), which is an inhibitor for GSK-3α and GSK-3β with an IC50 of 10 nM and 6.7 nM, respectively; Its inhibitory effect on GSK-3α and GSK-3β is 500 times stronger than on CDC2, ERK2 and other kinases;

CHIR-99021 (CT99021) HCl, which is the hydrochloride salt of CHIR-99021, is an inhibitor for GSK-3α/β with an IC50 of 10 nM/6.7 nM in cell-free assays, and can be used to distinguish GSK-3 and its closest homologues Cdc2 and ERK2;

BIO, which is a specific GSK-3 inhibitor, and has an IC50 of 5 nM for GSK-3α/β in cell-free assays;

IM-12, which is a selective GSK-3β inhibitor with IC50 of 53 nM, and enhances the Wnt signaling pathway;

TWS119 which is a GSK-3β inhibitor, and has an IC50 of 30 nM in cell-free assay;

1-Azakenpaullone, which is a highly selective GSK-3β inhibitor with IC50 of 18 nM;

CHIR-98014 which is a potent inhibitor for GSK-3α/β, and has an IC50 of 0.65 nM/0.58 nM in cell-free assays;

Tideglusib, which is an irreversible, non-ATP-competitive GSK-3β inhibitor, and has an IC50 of 60 nM in cell-free assays;

AR-A014418, which is an ATP-competitive and selective GSK3β inhibitor, and has an IC50 of 104 nM and a Ki of 38 nM in cell-free assays;

LY2090314, which is a potent GSK-3 inhibitor for GSK-3α/β with an IC50 of 1.5 nM/0.9 nM;

SB216763, which is a potent, selective inhibitor for GSK-3α/β with an IC50 of 34.3 nM;

AZD1080, which is an orally bioavailable and selective GSK3 inhibitor that is permeable to the brain, inhibits human GSK3α and GSK3β with a Ki of 6.9 nM and 31 nM, respectively, and is over 14 times more selective on GSK3 than on CDK2, CDK5, CDK1 and Erk2.

As a preferred embodiment of the present disclosure, the GSK3β inhibitor is CHIR-99021, also called CT99021; its molecular structural formula is shown by the following formula (I):

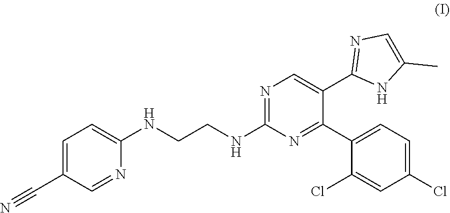 [SMILES: Cc1cnc(-c2cnc(NCCNc3ccc(C#N)cn3)nc2-c2ccc(Cl)cc2Cl)[nH]1]

As used in the present disclosure, the “TGFβ inhibitor” refers to a generic term for inhibitors capable of inhibiting the TGFβ signaling pathways in cells, including but not limited to: SB431542, A83-01, SB525334, LY2109761, RepSox and other TGFβ inhibitors of the same type, have the same function or engage the same target:

SB-431542, which is a potent and selective ALK5 inhibitor with an IC50 of 94 nM, and is 100 times more inhibitory on ALK5 than on p38, MAPK and other kinases;

A83-01, which is an inhibitor for ALK5, ALK4 and ALK7 with an IC50 of 12, 45, and 7.5 nM, respectively;

SB525334, which is a potent and selective TGFβ receptor I (ALK5) inhibitor with an IC50 of 14.3 nM in cell-free assays; its inhibitory effect on ALK4 is 4 times lower than on ALK5, and has no effect on ALK2, ALK3 and ALK6;

LY2109761, which is a novel and selective TGF-β receptor type I/II (TβRI/II) dual inhibitor with a Ki of 38 nM and 300 nM, respectively, in cell-free assays;

RepSox, which is a potent and selective TGFβR-1/ALK5 inhibitor, and influences the binding of ATP to ALK5 and ALK5 autophosphorylation with an IC50 of 23 nM and 4 nM, respectively, in cell-free assays;

SD-208, which is a selective TGF-βRI (ALK5) inhibitor with an IC50 of 48 nM, and is 100 times or above more selective on TGF-βRI than on TGF-βRII;

GW788388, which is a potent and selective ALK5 inhibitor with an IC50 of 18 nM in cell-free assays, also inhibits the activity of TGF-β type 11 receptors and activin type II receptors, but does not inhibit the activity of BMP type 11 receptors;

Galunisertib (LY2157299) is a potent TGFβ receptor I (TβRI) inhibitor with an IC50 of 56 nM in a cell-free assay;

SB505124, which is a selective TGFβR inhibitor for ALK4 and ALK5 with an IC50 of 129 nM and 47 nM, respectively, in cell-free assays, also inhibits ALK7, but does not inhibit ALK1, ALK2, ALK3 or ALK6; and;

EW-7197, which is a highly potent, selective and orally bioavailable TGF-beta receptor ALK4/ALK5 inhibitor with an IC50 of 13 nM and 11 nM, respectively.

As a preferred embodiment of the present disclosure, the TGFβ inhibitor is SB 431542 (or referred to as SB-431542), which has a molecular structure as shown in the following formula (II):

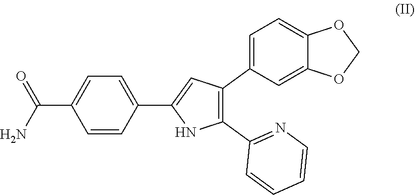 [SMILES: NC(=O)c1ccc(-c2cc(-c3ccc4c(c3)OCO4)c(-c3ccccn3)[nH]2)cc1]

As a preferred embodiment of the present disclosure, the TGFβ inhibitor is A83-01 (or referred to as A8301), which has a molecular structure as shown in the following formula (III):

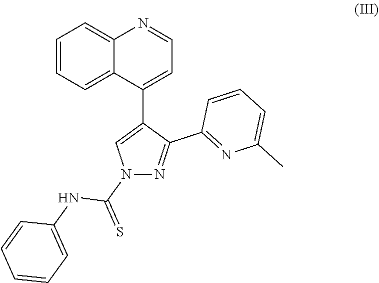 [SMILES: Cc1cccc(-c2nn(C(=S)Nc3ccccc3)cc2-c2ccnc3ccccc23)n1]

G9a histone methyltransferase (G9aHMTase) is also called euchromatic histone-lysine N-methyltransferase 2 (EHMT2). As used in the present disclosure, the “G9aHMTase inhibitor” refers to a general term of the inhibitors that can inhibit G9aHMTase in a cell, including but not limited to: BIX01294, UNC0638, A-366, UNC0631, BRD4770, UNC0224, UNC0646, UNC0642, and other G9aHMTase inhibitors or compounds of the same type, have same function, or engage the same target:

BIX01294, which is a G9a histone methyltransferase inhibitor with an IC50 of 2.7 μM in a cell-free assay, reduces most of the histone H3K9me2, and also weakly inhibits GLP (mainly H3K9me3), but has no significant inhibitory effect on other histone methyltransferase; BIX01294 specifically inhibits the activity of HMTs responsible for methylating the H3K9 site;

UNC0638 is an effective and selective histone lysine methyltransferase (HMTase) inhibitor, which acts on G9a and GLP with IC50 of <15 nM and 19 nM, respectively, and effectively and selectively acts on a variety of epigenetics and non-epigenetic targets; UNC0638 specifically inhibits the activity of HMTs responsible for methylation of the H3K9 site;

A-366 is an effective and selective G9a/GLP histone methyltransferase inhibitor with IC50 of 3.3 nM; its selectivity with G9a/GLP is over 1000 times higher than with 21 other methyltransferases;

UNC0631 is a potent histone methyltransferase G9a inhibitor with IC50 of 4 nM;

BRD4770 is a histone methyltransferase G9a inhibitor with an IC50 of 6.3 μM and induces cell death. BRD4770 is a novel histone methyltransferase G9a (EHMT2) inhibitor with an EC50 value of 5 uM (trimethylated H3K9) in PANC-1 cells;

UNC0224 is a G9a HMTase inhibitor with IC50 of 15 nM;

UNC0646 is a highly selective inhibitor of histone lysine methyltransferases G9a and GLP (IC50 values for G9a and GLP are 6 nM and 15 nM, respectively). UNC0646 can also effectively block the activity of G9a/GLP methyltransferase in cells (IC50=10 nM in MCF7 cells) while exhibiting lower cytotoxicity (EC50=4.7 μM in MCF7 cells). In vivo, researchers have previously found other highly selective G9a/GLP inhibitors, including the cytochemical probe UNC0638, which is superior in terms of functional potential and cytotoxicity. However, UNC0638 has poor pharmacokinetic (PK) characteristics and is not suitable for animal experiments;

UNC0642 is an effective and selective G9a/GLP inhibitor, which inhibits histamine H3 receptor and sigma-2 receptor, with Ki of 45 nM and 900 nM, respectively. UNC0642 was found to not only have higher in vitro and cellular potency, low cytotoxicity, preferential selectivity, but also exhibits better in vivo PK characteristics, making it more suitable for animal experiments.

As a preferred embodiment of the present disclosure, the G9aHMTase inhibitor is BIX01294 (or BIX-01294), which has a molecular structure as shown in the following formula (IV):

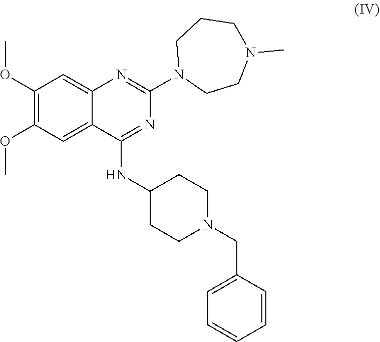 [SMILES: COc1cc2nc(N3CCCN(C)CC3)nc(NC3CCN(Cc4ccccc4)CC3)c2cc1OC]

The present disclosure also includes compounds, pharmaceutical drugs, analogs and/or salts, hydrates or precursors equivalent to the above-mentioned compounds I, II or III, IV; and also includes naturally occurring and artificially synthesized compounds thereof.

Analogs of the compounds include, but are not limited to, isomers and racemates of the compounds. Compounds have one or more asymmetric centers. Therefore, these compounds can exist as racemic mixtures, individual enantiomers, individual diastereomers, diastereomeric mixtures, cis or trans isomers.

The “salts” include, but are not limited to: (1) salts formed with such inorganic acids as hydrochloric acid, sulfuric acid, nitric acid, phosphoric acid, etc.; and (2) salts formed with such organic acids as acetic acid, oxalic acid, succinic acid, tartaric acid, methanesulfonic acid, maleic acid, arginine or the like. Other salts include salts formed with alkali or alkaline earth metals such as sodium, potassium, calcium or magnesium, etc.

The “precursor of a compound” refers to a precursor that can be converted into any of the above compounds in a medium after the precursor is applied or treated in a suitable manner, or to a salt or solution formed with any of the above compounds.

In the composition of the present disclosure, the GSK3 β inhibitor, the TGF β inhibitor, and the G9aHMTase inhibitor are present in weight part ratio of (5-80):(0.1-50):(0.1-50); preferably, (10-70):(0.5-40):(0.5-40); or in a solution state at molar concentration ratio: (0.1-20):(0.01-20):(0.01-20); preferably, (0.5-10):(0.05-10):(0.05-10).

The composition of this disclosure is used for chemically inducing human fibroblasts to transdifferentiate into hepatocytes; it can also be used for the development or preparation of a drug or prodrug (or drug formula) for treating hepatic fibrosis (cirrhosis), pulmonary fibrosis and fibrosis diseases of other organ or tissue.

There is no particular limitation on the dosage form of the composition of the present disclosure, and it may be any dosage form suitable for mammal administration; the dosage forms that can be prepared include: powders, pulvis, tablets, pills, capsules, sustained release dosages, controlled release dosages and other solid dosage forms; injections, infusions, suspensions and other liquid dosage forms, as well as gas dosage forms, semi-solid dosage forms and other dosage forms. Preferably, the dosage form may be, but is not limited to, a solid dosage form such as a powder, a granule, a capsule, a sustained release agent, a tablet, or a liquid dosage form such as an injection, an infusion, a solution, or a suspension.

The preparation method of the composition of the present disclosure is determined according to the dosage form required to be prepared and the administration route. Those skilled in the art can prepare the present composition according to common preparation method of pharmaceutical composition according to the combination and ratios provided herein.

It should be understood that although in the specific embodiment, the present inventor only lists several composition forms, but those skilled in the art can also deduce from this that any other composition form of the present disclosure should also has outstanding effects.

The inventors have demonstrated for the first time that the small molecule composition of the present disclosure can be used to develop and prepare drugs or prodrugs or pharmaceutical formulations that prevent, ameliorate or treat hepatic fibrosis (cirrhosis), pulmonary fibrosis, and fibrosis diseases of other organ or tissue. When used to prevent, ameliorate, or treat hepatic fibrosis (cirrhosis), pulmonary fibrosis, and fibrosis diseases of other organ or tissue, the effective dose of the composition used may vary with the mode of administration and the type of fibrotic disease to be treated and the disease severity. The specific situation is determined according to the situation of the individual subject, which is within the range of judgement of skilled physicians or pharmacists.

In the present disclosure, the fibroblasts include, but are not limited to, fibroblasts from different tissues or organs of the human body, such as skin fibroblasts, hepatic fibroblasts (hepatic stellate cells), lung fibroblasts, renal fibroblasts, pancreatic fibroblasts, and fibroblasts from other tissues or organs in the body. Preferred are human skin fibroblasts and hepatic fibroblasts (hepatic stellate cells).

Medium and Reagents

This disclosure also provides a culture medium for direct reprogramming (transdifferentiation) of human fibroblasts into hepatocytes by the small molecule composition (hereinafter referred to as fibroblast to hepatocyte transdifferentiation culture medium).

According to the formulation of the composition in final concentrations provided by the present disclosure, small molecule composition with specific final concentration can be selected for medium preparation. As a preferred embodiment of the present disclosure, different components in the specific small molecule composition are respectively dissolved in DMSO (dimethyl sulfoxide) or other organic solvents or aqueous solvents according to different properties of the solutes and their different solubilities to form concentrated liquid reagent (ranging from 1:50-1:10,000); then, according to the final concentration requirement of the specific small molecule composition, concentrates of respective small molecule's organic solution is diluted and added to basal cell culture medium containing 10% calf serum (or a serum-free medium comprising various cytokines or growth factors), to obtain the fibroblast to hepatocyte transdifferentiation medium, wherein, the content in percentage of each component in the medium can also be increased or decreased by 50%, preferably by 30%, more preferably by 20%, such as 10% and 5% (the percentages are in v/v).

As a preferred embodiment of the present disclosure, the basal cell culture medium includes, but is not limited to, DMEM/F12, MEM, DMEM, F12, IMDM, RPMI1640, Neuronal basal, or Fischers, etc., which are all commercially available products.

As a preferred embodiment of the present disclosure, the “serum-free medium” refers to a cell culture medium without serum but with various nutritional components (such as growth factors, tissue extracts, etc.) that support cell proliferation and biological activities. That is, additives of various cytokines or growth factors other than serum can be added to a cell culture medium composed of basal cell culture medium.

As a preferred embodiment of the present disclosure, the serum-free medium contain various cytokines or growth factors, including but not limited to, ITS, N2, B27, and the like, all of which can be self-formulated or commercially available products.

It should be understood that those skilled in the art are familiar with the formulation or purchase route of the basal cell culture medium or serum-free medium, and therefore, the basal cell culture medium or serum-free medium is not limited to those exemplified in the present disclosure.

As a preferred embodiment of the present disclosure, the “fibroblast to hepatocyte transdifferentiation culture medium” is specifically formulated as follows:

(1) Mixing \textcircled{1} GSK3 β inhibitor (or GSK3 β inhibitor CHIR-99021): the final concentration is 0.1-20 uM; the preferred amount is 0.5-10 uM; \textcircled{2} TGF β inhibitor (or TGF β inhibitor SB431542 or/and A83-01): the final concentration is 0.01-20 uM; the preferred amount is: 0.05-10 uM; and \textcircled{3} G9aHMTase inhibitor (or G9a HMTase inhibitor BIX01294): the final concentration is 0.01-20 uM; the preferred amount is: 0.05-10 uM, to obtain the small molecule composition for chemically inducing human fibroblasts to directly reprogram (transdifferentiate) into hepatocytes.

(2) Subject each of the above small molecules to the following process: dissolving to formulate a concentrated solution→diluting into a basal cell culture medium→mixing, to prepare “fibroblast to hepatocyte transdifferentiation culture medium”.

This disclosure also provides reagents for injection or oral use in laboratory animals for chemically inducing human fibroblasts to directly reprogram (transdifferentiate) into hepatocytes.

As a preferred embodiment of the present disclosure, corresponding dosage of each small molecule composition in any of the foregoing compositions is calculated based on kilogram body weight, and is dissolved in solution of Captisol (1-30%), Tween-80 (5%) to obtain the reagents for injection or oral use in laboratory animals. Captisol (1-30%) is preferred.

Culturing Method

This disclosure also discloses a method for inducing human fibroblasts to directly reprogram (transdifferentiate) into hepatocytes by a small molecule composition, comprising the following steps:

(1) Formulation of concentrated liquid reagent: According to any of the foregoing compositions, each component is dissolved in an organic solvent or an aqueous solvent to prepare a concentrated liquid reagent; preferably, the organic solvent includes dimethyl sulfoxide; more preferably, the aqueous solvent includes: water, physiological saline, phosphate buffer solution;

(2) Obtaining culture medium: Dilute the concentrated solution reagent in step (1) into a basal cell culture medium containing 5-20% calf serum or a serum-free medium comprising a variety of cytokines or growth factors (so that the concentration of each component conforms to the final concentration defined in any of the foregoing compositions), to obtain a medium for inducing fibroblasts to transdifferentiate into hepatocytes;

Wherein, the content in percentage of each component in the medium can also be increased or decreased by 50%, preferably by 30%, more preferably by within 20%, such as 10% and 5%.

(3) Inducing fibroblasts to transdifferentiate into hepatocytes: suspend fibroblasts in a basal cell culture medium comprising 5-20% calf serum or a serum-free medium comprising various cytokines or growth factors and plate them, after the cells have adhered, replace basal medium with the transdifferentiation medium of step (2), culture at 37° C., and change the medium every 2-4 days; the cells are passaged every 3-15 days.

(4) Passaging of hepatocytes derived from fibroblasts transdifferentiation: Discard the original culture solution, wash the cells once with PBS, add cell digestion solution to digest the cells at 37° C. for 1-5 min, then stop cell digestion, centrifuge and discard the supernatant. Resuspend cell pellets and plate them at 1:1-1:3 passage. Cells should be cultured according to the method above using the transdifferentiation medium of step (2), and solution should be changed every 2-4 days. The digestive solution used includes trypsin, EDTA, Acutase, TrypleE, etc. Passage cells every 3-15 days.

(5) Harvesting the hepatocytes derived from fibroblasts transdifferentiation: After culturing and passaging the cells according to experiment steps (3) and (4) mentioned above for 2-4 weeks. Fibroblasts can complete hepatic transdifferentiation, and transdifferentiated hepatocytes can be obtained. The hepatocytes can be used for other scientific research experiments; detection and evaluation of new drug toxicity and efficacy; and providing hepatocyte source for the construction of biological artificial liver and clinical cell transplantation. Culturing of the fibroblast-transdifferentiated hepatocytes is the same as the experiment steps of the above-mentioned culture method.

Functional assay of fibroblast-transdifferentiated hepatocytes: Culture fibroblast-transdifferentiated hepatocytes as described above, and use hepatocytes harvested at different timepoints of culture for functional assay.

The present small molecule composition for chemically inducing fibroblasts directly reprogram (transdifferentiate) into hepatocytes and the culture medium and reagents prepared thereby, as well as the experiment method and the obtained fibroblast-transdifferentiated hepatocytes not only can be used to develop and prepare drug or prodrug for clinical treatment of hepatic fibrosis (cirrhosis), pulmonary fibrosis and fibrosis disease of other organs or tissues, but also can be widely used as prevention methods and mechanism study for fibrosis in tissues or organs; as well as constructing cell model for liver disease research, pharmacology and toxicological safety testing. The obtained fibroblast-transdifferentiated hepatocytes can be used continuously for functional assay and preclinical studies. This method not only provides a new option for the prevention and treatment of hepatic fibrosis (cirrhosis), pulmonary fibrosis and fibrosis diseases in other organs or tissues, but also provides a new cell source for the pharmaceutical, clinical and scientific applications of hepatocytes. With a wide range of prospects for application, the present disclosure enriches the theory of stem cell reprogramming and expands its application possibilities, and thus has great scientific significance and practical value.

The benefits of the present disclosure are as follows:

1. Referring to previous studies of cell reprogramming, first proposes innovative theory that: by selecting a composition of induction factors that can construct and maintain the unique gene expression profile or biological behaviors of the target cell, and break various “energy barriers” during the reprogramming process, fibroblasts can be induced to directly transdifferentiated into defined target cells;

2. For the first time, use only combinations of small molecules to induce fibroblasts to directly reprogram (transdifferentiate) into hepatocytes, thus providing a new source of hepatocytes for scientific, medical, and clinical applications, such as hepatocyte transplantation for treatment of hepatic failure;

3. In the present disclosure, the small molecules used are stable in nature, and can be easily manipulated through action time, dosage and ways of combination; their effects are also stable and consistent;

4. The present disclosure excludes introduction of exogenous genes (transcription factors), or making changes to cell's genetic sequence, and thus avoids new carcinogenic risks caused by the introduction of exogenous genes or genetic changes, therefore this method is safe, reliable, and suitable for clinical application;

5. First uses small molecule combination to induce fibroblasts to reprogram directly into hepatocytes without going through the induced pluripotent stem cell (iPSC) stage, thus avoiding the risk of carcinogenesis by pluripotent stem cells; small molecules are also easy to apply for inducing in situ cell transdifferentiation in vivo, and can be developed into pharmaceutical drugs;

6. the fibroblasts in this method can be obtained from the patient and transdifferentiate into autologous hepatocytes, which have two major advantages: first of all, they can be applied clinically as they can minimize or avoid immune rejection risk in hepatocyte transplantation; secondly, they can be used for building crowd-representative hepatocyte bank for liver toxicity and efficacy screening of new drugs; therefore, the present disclosure can provide new cell sources for clinical and medical applications;

7. Chemical small molecules are stable in nature and suitable for development into pharmaceutical drugs. The method is easy to apply in vivo to alleviate or reverse hepatic fibrosis (cirrhosis), or fibrosis of other organs or tissues. The present disclosure can be used to develop or prepare new drugs or prodrugs for treating human hepatic fibrosis (cirrhosis), pulmonary fibrosis and fibrosis diseases of other organ or tissue. It can also provide new ideas, new methods, and new measures for the clinical treatment of fibrotic diseases; and creates a new research field for various fibrotic diseases.

8. The small molecule combination can be prepared into medium or reagents for chemically inducing fibroblasts to transdifferentiate into hepatocytes for the purpose of scientific research; it can provide medium or kits for the study of relevant mechanisms of hepatocyte transdifferentiation and liver disease research;

9. The small molecule composition of the present disclosure and the method for chemically inducing fibroblasts to transdifferentiate into hepatocytes are simple, requiring few experiment steps, inexpensive, and easy to produce. The transdifferentiated hepatocytes not only can provide source of hepatocytes for scientific research and medical applications, but also can be easily used in clinical application, and can promote the development of related biomedical industries.

The present disclosure is further illustrated with reference to the specific examples below. It should be understood that these examples are only for illustrating the present disclosure and are not intended to limit the scope of the present disclosure. The experimental methods that do not specify the specific conditions in the following examples are generally performed according to conventional conditions such as those described in [US] J S Bonifensnon et al.; translated by Zhang Jingbo, Fang Jin, Wang Haijie, etc., “Short Protocols in Cell Biology, Science Press 2007; or as recommended by the manufacturer.

### Example 1. Small Molecule Composition Inducing Human Fibroblasts to Directly Reprogram (Transdifferentiate) into Hepatocytes, and the Formulation of the Medium and Reagents

The following compositions or culture mediums are designed, and can be formulated at molar concentration or weight concentration:

1. Formulation of Small Molecule Composition for Fibroblast Transdifferentiation into Hepatocytes

(1) Composition 1 for Fibroblast Transdifferentiation into Hepatocyte

GSK3β inhibitor CHIR-99021: final concentration 5 uM;

TGFβ inhibitor SB431542: final concentration 2 uM;

G9aHMTase inhibitor BIX01294: final concentration 3 uM;

(2) Composition 2 for Fibroblast Transdifferentiation into Hepatocyte

GSK3β inhibitor CHIR-99021: final concentration 6 uM;

TGFβ inhibitor SB431542: final concentration 0.5 uM;

G9aHMTase inhibitor BIX01294: final concentration 5 uM;

(3) Composition 3 for Fibroblast Transdifferentiation into Hepatocyte

GSK3β inhibitor CHIR-99021: final concentration 3 uM;

TGFβ inhibitor A83-01: final concentration 1 uM;

G9aHMTase inhibitor BIX01294: final concentration 2 uM;

(4) Composition 4 for Fibroblast Transdifferentiation into Hepatocyte

GSK3β inhibitor BIO: final concentration 1 uM;

TGFβ inhibitor A83-01 final concentration 1 uM;

G9aHMTase inhibitor UNC0638: final concentration 3 uM;

(5) Composition 5 for Fibroblast Transdifferentiation into Hepatocyte

GSK3β inhibitor CHIR-99021: final concentration 3 uM;

TGFβ inhibitor SB431542: final concentration 5 uM;

G9aHMTase inhibitor UNC0638: final concentration 5 uM;

(6) Composition 6 for Fibroblast Transdifferentiation into Hepatocyte

GSK3β inhibitor BIO: final concentration 3 uM;

TGFβ inhibitor SB431542: final concentration 2 uM;

G9aHMTase inhibitor BIX01294: final concentration 2.5 uM;

(7) Composition 7 for Fibroblast Transdifferentiation into Hepatocyte

GSK3β inhibitor CHIR-99021: final concentration 5 uM;

TGFβ inhibitor LY2109761: final concentration 2 uM;

G9aHMTase inhibitor BIX01294: final concentration 2 uM;

(8) Composition 8 for Fibroblast Transdifferentiation into Hepatocyte

GSK3β inhibitor CHIR-99021: final concentration 4 uM;

TGFβ inhibitor SB431542: final concentration 3 uM;

G9aHMTase inhibitor BIX01294: final concentration 2 uM;

(9) Composition 9 for Fibroblast Transdifferentiation into Hepatocyte

GSK3β inhibitor TWS119: final concentration 4 uM;

TGFβ inhibitor A83-01: final concentration 2 uM;

G9aHMTase inhibitor BIX01294: final concentration 5 uM;

(10) Composition 10 for Fibroblast Transdifferentiation into Hepatocyte

GSK3β inhibitor CHIR-98014: final concentration 2 uM;

TGFβ inhibitor SB431542: final concentration 5 uM;

G9aHMTase inhibitor BIX01294: final concentration 5 uM;

Each specific small molecule composition is first dissolved in DMSO according to the aforementioned “culture method” step (1) to prepare a concentrated liquid reagent.

2. Preparation of Medium for Fibroblast Transdifferentiation into Hepatocyte

The DMSO concentrated liquid reagents of each component of composition 1-10 for transdifferentiation of fibroblasts into hepatocytes from step 1 is formulated according to the aforementioned “Culturing method” step (2) (the selected basic cell culture medium is DMEM) to obtain Culture Medium 1-10 for transdifferentiation of fibroblasts into hepatocytes (that is, the final compound concentrations in medium 1 and the final concentration of composition 1 are the same, the final compound concentrations in medium 2 and the final concentration of composition 2 are the same, . . . , the final compound concentrations in medium 10 and the final concentration of composition 10 are the same).

3. Formulation of Oral Reagent for Fibroblast Transdifferentiation

The DMSO concentrated solution of the composition 10 for fibroblast transdifferentiation into hepatocyte is dissolved in 5% Captisol to prepare an oral reagent for fibroblast transdifferentiation (same concentration as the final concentration of compound of composition 10).

### Example 2: Fibroblast to Hepatocyte Transdifferentiation Culture Medium 6 for Induces Transdifferentiation of Fibroblast into Hepatocytes

1. Culturing of Fibroblast-Transdifferentiated Hepatocyte

Fibroblasts are suspended, plated in basal cell medium DMEM containing 10% calf serum, and cultured at 37° C.

After the fibroblasts adhere to the wall, the original medium is discarded and replaced with fibroblast to hepatocyte transdifferentiation Culture Medium 6; culture cells at 37° C., replaced medium every 3 days, and passage the cells once every 7 days.

2. Subculture of Fibroblast Transdifferentiated Hepatocytes

Passaging steps: Discard the original culture solution, wash the cells once with PBS, add cell digestion solution to digest the cells at 37° C. for 3 min, then stop cell digestion, centrifuge and discard the supernatant. Resuspend cell pellets and plate them at the ratio of 1:2. Cells should be cultured with fibroblast to hepatocyte transdifferentiation Culture Medium 6, and solution is changed every 4 days. The digestive solution used includes trypsin, EDTA, Acutase, TrypleE, etc. Passage cells every 3-15 days. The digestive solution used is pancreatin (also can use EDTA, Acutase, TrypleE) and the like. Passage every 5 days.

3. Harvesting of Transdifferentiated Hepatocytes

After the culture and subculture of the fibroblast transdifferentiated hepatocytes through the above experiment steps 1-2 for 2-4 weeks, transdifferentiated hepatocytes can be obtained, which can be used for other scientific research experiments.

4. Morphological Comparison of Human Fibroblasts (HFF) and Transdifferentiated Hepatocytes (ciHep)

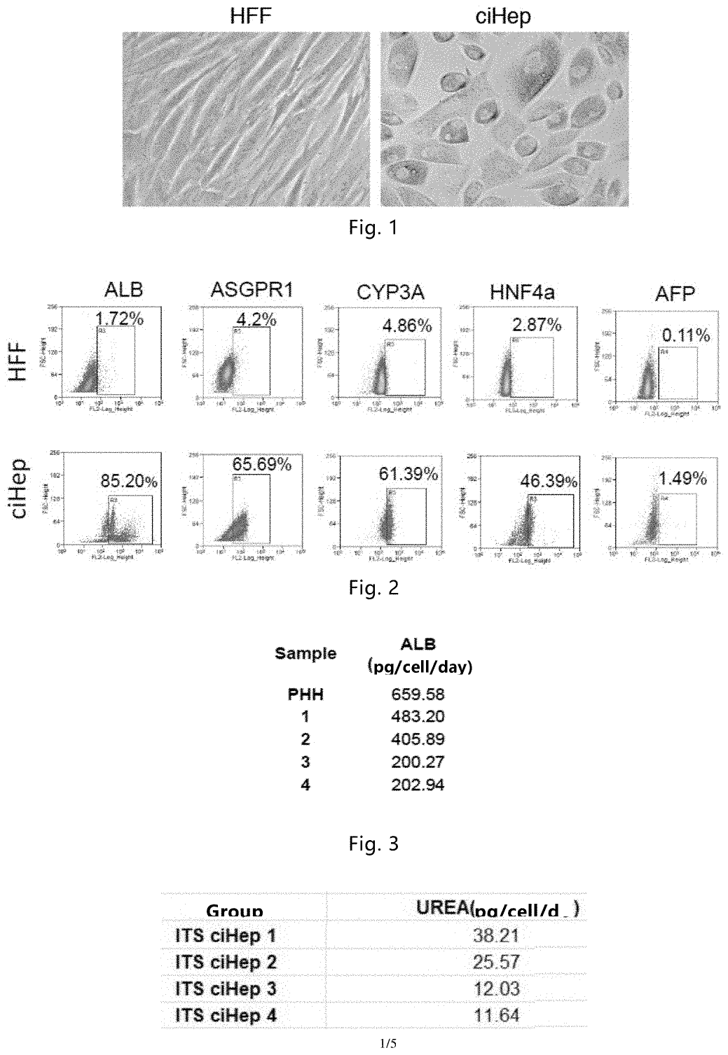

Morphological comparison between human fibroblasts and the hepatocytes obtained from fibroblast transdifferentiation in step 3, results are shown in FIG. 1.

The experiment results in FIG. 1 show that after human fibroblasts are transdifferentiated into hepatocytes, the morphology changes significantly, which is consistent with the morphology of hepatocytes.

### Example 3: Flow Cytometric Analysis of Liver-Specific Marker Staining of the Human Fibroblast-Transdifferentiated Hepatocyte-Like Cells Induced by Fibroblast to Hepatocyte Transdifferentiation Culture Medium 1

The method of inducing the transdifferentiation of human fibroblasts into hepatocyte by fibroblast to hepatocyte transdifferentiation Culture Medium 1 is the same as in Example 2.

fibroblast-transdifferentiated hepatocyte-like cells are subjected to flow cytometric analysis of liver-specific marker staining. Using conventional immunostaining methods, the hepatocyte-like cells obtained from the above experiment steps are subjected to immunostaining of human hepatocyte-specific markers (AAT, ALB, Asgpr, CYP3A, HNF4a). The immunostaining method is as follows:

(1) Discard the cell Culture Medium and rinse the cells once with PBS;

(2) Digest with 0.05% trypsin at 37° C. for 5 minutes, trypsin is terminated with a trypsin terminator, or a cell Culture Medium containing serum/albumin, centrifuge at 800-1000 rpm for 3-5 minutes, and discard the supernatant;

(3) After fixing with 2% paraformaldehyde for 10 minutes, rinse with PBS for 5 minutes×3 times;

(4) Block with 10% sheep serum at room temperature for 60 minutes;

(5) 0.1% Triton: 5-10 min;

(6) Treat with primary antibody (rabbit anti-ALB antibody, mouse anti-CYP3A antibody or rabbit anti-Asgpr antibody) at room temperature for 1 hour or overnight at 4° C.

(7) Rinse with PBS for 5 minutes×3 times;

(8) Treat with secondary antibody (Cy3-labeled goat anti-rabbit antibody, FITC-labeled goat anti-mouse or goat anti-rabbit antibody) at room temperature for 45-60 min;

(9) Wash in PBS for 5 min×3 times.

Flow cytometry is then performed and the results are shown in FIG. 2.

The experiment results shown in FIG. 2 show that the percentage of cells with positive hepatocyte-specific markers among the fibroblast-transdifferentiated hepatocyte-like cells is very high, while the embryonic hepatocyte marker AFP is practically unexpressed. Therefore, these results prove that the transdifferentiation medium and the culture method of the present disclosure can transdifferentiate human fibroblasts into mature human hepatocytes.

### Example 4: The Comparison of Albumin Production Between the Fibroblast-Transdifferentiated Hepatocytes by the Induction of Fibroblast to Hepatocyte Transdifferentiation Culture Medium 2, 3, 4, 5, and Primary Hepatocyte

The method for the induction of fibroblast transdifferentiation into hepatocyte with fibroblast to hepatocyte transdifferentiation Culture Medium 2, 3, 4, 5 (the treatment groups are sequentially grouped into group 1, 2, 3, and 4) is the same as in Example 2. The 4 groups of fibroblast-transdifferentiated hepatocyte-like cells were compared with human primary hepatocytes (PHH) for albumin production. The specific method is:

The albumin secretion assay was performed on the four groups of fibroblast-transdifferentiated hepatocyte-like cells from the above experiment steps and the commercially available human primary hepatocytes using an ELISA kit. The assay steps refer to the instruction of the kit (Bioassay System Corporation/DIAG-250, BCG Albumin assay kit).

The comparison results of albumin production between the four groups of fibroblast-transdifferentiated hepatocyte-like cells and human primary hepatocytes (PHH) are shown in FIG. 3.

The experiment results in FIG. 3 show that the fibroblast-transdifferentiated hepatocyte-like cells have the function of albumin production, which is unique to human hepatocytes.

### Example 5: Urea Production Functional Assay of Fibroblast-Transdifferentiated Hepatocyte-Like Cells by the Induction of Fibroblast to Hepatocyte Transdifferentiation Culture Mediums 6, 7, 8, and 9

The method for the induction of fibroblast transdifferentiation into hepatocyte with fibroblast to hepatocyte Culture Medium 6, 7, 8, and 9 (the treatment groups are sequentially grouped into group 1, 2, 3, and 4) is the same as in Example 2. The 4 groups of fibroblast-transdifferentiated hepatocyte-like cells were compared with human primary hepatocytes (PHH) for urea production. The specific method is:

Urea nitrogen test kit was used to test urea synthesis function on transdifferentiated hepatocyte-like cells obtained from the above experiments and commercially available human primary hepatocytes; The test steps can refer to the instructions of the kit (Bioassay System/DIUR-500, Urea assay kit);

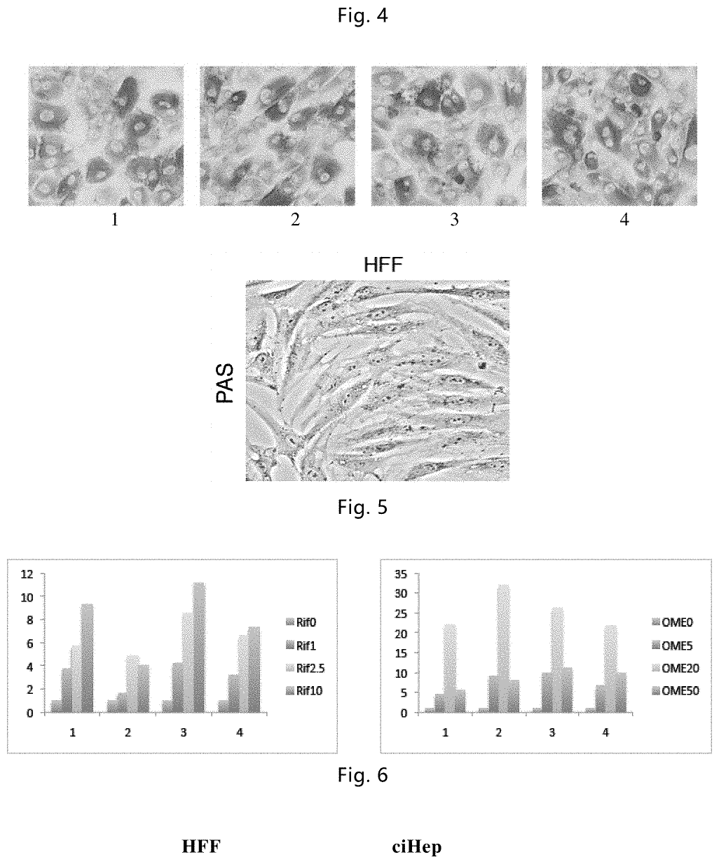

The source of urea is blood ammonia in the blood, and the amount of urea produced reflects the detoxification ability of hepatocyte. The results of urea production of fibroblast-transdifferentiated hepatocyte-like cells of the four groups are shown in FIG. 4.

The source of urea is blood ammonia in the blood, and the amount of urea produced reflects the detoxifying ability of hepatocytes.

The experiment results shown in FIG. 4 prove that the fibroblast-transdifferentiated hepatocytes have the function of urea production unique to hepatocytes.

### Example 6, Glycogen Staining of Fibroblast-Transdifferentiated Hepatocyte-Like Cells Induced by Fibroblast to Hepatocyte Transdifferentiation Culture Medium 6, 7, 8 and 9

The method for the induction of fibroblast transdifferentiation into hepatocyte with fibroblast to hepatocyte Culture Medium 6, 7, 8, and 9 (the treatment groups are sequentially grouped into group 1, 2, 3, and 4) is the same as in Example 2.

Glycogen staining was performed on human fibroblast-transdifferentiated hepatocyte-like cells. The degree of staining demonstrates the ability of hepatocytes to store glycogen. Schiff method was used for liver glycogen staining. The specific method is:

(1) Discard cell culture medium, rinse once with PBS;

(2) After fixed with 4% paraformaldehyde for 10 minutes, rinse with PBS for 5 minutes×3 times;

(3) Add PAS-I solution for 10 min and rinse under running water;

(4) Add PAS-II solution for 1-2 min and rinse under running water;

(5) Take pictures with a microscope.

Glycogen staining results of human fibroblasts transdifferentiated hepatocyte-like cells are shown in FIG. 5; glycogen staining results of hepatocyte-like cells by induction of media 6, 7, 8, and 9 are listed in the upper panel from left to right as 1, 2, 3, and 4.

The experiment results shown in FIG. 5 show that hepatocyte-like cells obtained by transdifferentiation of fibroblast is positive for liver glycogen staining, while fibroblasts in lower panel have negative liver glycogen staining. This proves that the fibroblast-transdifferentiated hepatocyte-like cells obtained by the method of the present disclosure have the same glycogen storage activity as human hepatocytes.

### Example 7. The P450 Enzyme (CYP3A4 and CYP1A2) Activity Induction of Fibroblast Transdifferentiated Hepatocytes Induced by Fibroblast to Hepatocyte Transdifferentiation Culture Medium 6, 7, 8, 9

The method for the induction of fibroblast transdifferentiation into hepatocyte with fibroblast to hepatocyte Culture Medium 6, 7, 8, and 9 (the treatment groups are sequentially grouped into group 1, 2, 3, and 4) is the same as in Example 2.

Inducing the P450 enzymes (CYP3A4 and CYP1A2) activity of fibroblast-transdifferentiated hepatocytes:

(1) Fibroblast-transdifferentiated hepatocytes obtained by the above experiment steps were found to have increased P450 enzyme (CYP3A4) activity under induction of rifampicin. The method for inducing P450 enzyme activity is as follows:

Treat cells with different concentrations of rifampicin (1 uM, 10 uM, 25 uM), and use cell group without the addition of rifampicin (but other conditions remain the same) as control. Change the medium every 24 hours, collect RNA from extracted cells at 48 hours, and measure the expression level of P450 enzyme CYP3A4 gene by Real-time PCR;

(2) The expression level of the P450 enzyme CYP1A2 gene in the hepatocyte-like cells obtained by the above experiment steps is tested after induction with omeprazole. The method of P450 enzyme activity induction is as follows:

Treat cells with omeprazole at different concentrations (1 uM, 10 uM, 25 uM), use the cell group without adding omeprazole (other conditions remain the same) as a control. The solution was changed every 24 hours, and the cells were collected for RNA extraction at 48 hours, and the expression level of P450 enzyme CYP3A4 gene was determined by Real-time PCR.

The results of P450 enzyme (CYP3A4 and CYP1A2) activity induction results are shown in FIG. 6 (Figure left: increased activity of CYP3A4 induced by different concentrations of rifampin; right: increased activity of CYP1A2 induced by different concentrations of omeprazole). In the figure, the P450 enzyme activity induction results of fibroblast-transdifferentiated hepatocyte-like cells obtained with fibroblast to hepatocyte transdifferentiation medium 6, 7, 8, and 9 are arranged from left to right as 1, 2, 3, and 4.

The experiment results in FIG. 6 show that the fibroblast-transdifferentiated hepatocyte-like cells obtained by the method of the present disclosure have P450 metabolic enzyme activity unique to hepatocytes. The y coordinate in the figure is the relative activity of the enzyme.

### Example 8: Fat Staining of the Fibroblast-Transdifferentiated Hepatocyte-Like Cells Induced by Fibroblast to Hepatocyte Transdifferentiation Culture Medium 8, the Degree of the Staining Shows the Ability of Hepatocytes to Metabolize Fat

The method for the induction of fibroblast transdifferentiation into hepatocyte with fibroblast to hepatocyte Culture Medium 8 is the same as in Example 2.

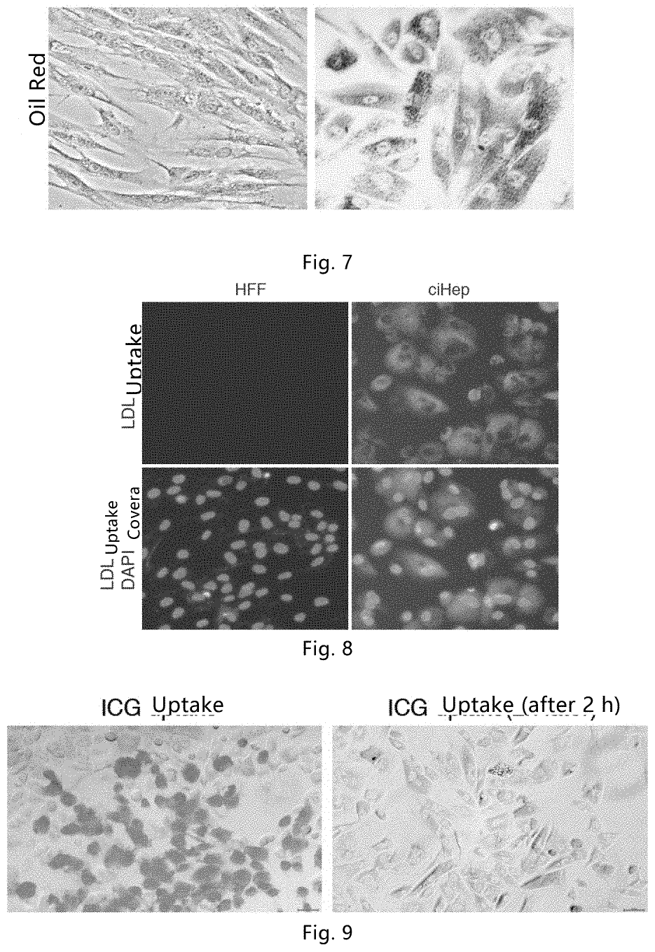

The conventional kit for fat staining is used, and the method steps are described in the kit. The staining results are shown in FIG. 7.

The experimental results in FIG. 7 show that the fibroblast-transdifferentiated hepatocytes have positive fat staining and the fibroblasts have negative fat staining; this proves that hepatocyte-like cells obtained by the method of the present disclosure have the ability to metabolize fat, which is unique to hepatocytes.

### Example 9. Fat Uptake of the Fibroblast-Transdifferentiated Hepatocyte-Like Cells Induced by Fibroblast to Hepatocyte Transdifferentiation Culture Medium 9, the Degree of the Staining Shows the Ability of Hepatocytes to Uptake Fat

The method for the induction of fibroblast transdifferentiation into hepatocyte with fibroblast to hepatocyte Culture Medium 9 is the same as in Example 2.

The conventional kit for fat uptake is used, and the method steps are described in the kit. The staining results are shown in FIG. 8.

The experiment results in FIG. 8 show that fibroblast-transdifferentiated hepatocyte-like cells obtained by the method of the present disclosure have fat uptake and storage functions of hepatocytes, but fibroblasts do not have such function.

### Example 10. ICG Uptake of Human Fibroblast Transdifferentiated Hepatocytes. The Degree of Staining Demonstrates the Ability of Hepatocytes to Take Up and Excrete Foreign Bodies

The method for the induction of fibroblast transdifferentiation into hepatocyte with fibroblast to hepatocyte Culture Medium 2 is the same as in Example 2.

The conventional kit for ICG uptake is used, and the method steps are described in the kit. The staining results are shown in FIG. 9.

The experiment results in FIG. 9 showed that fibroblast-transdifferentiated hepatocyte-like cells obtained by the method of the present disclosure have the ability of human hepatocytes to take up foreign bodies and excrete foreign bodies, proving that they have hepatocyte-related functions.

### Example 11: Comparison of Morphology and Function of Human Hepatic Fibroblast Cell Line Lx2 (Hepatic Stellate Cell, HSC) and the Hepatocytes Transdifferentiated Therefrom (Lx2-ciHep)

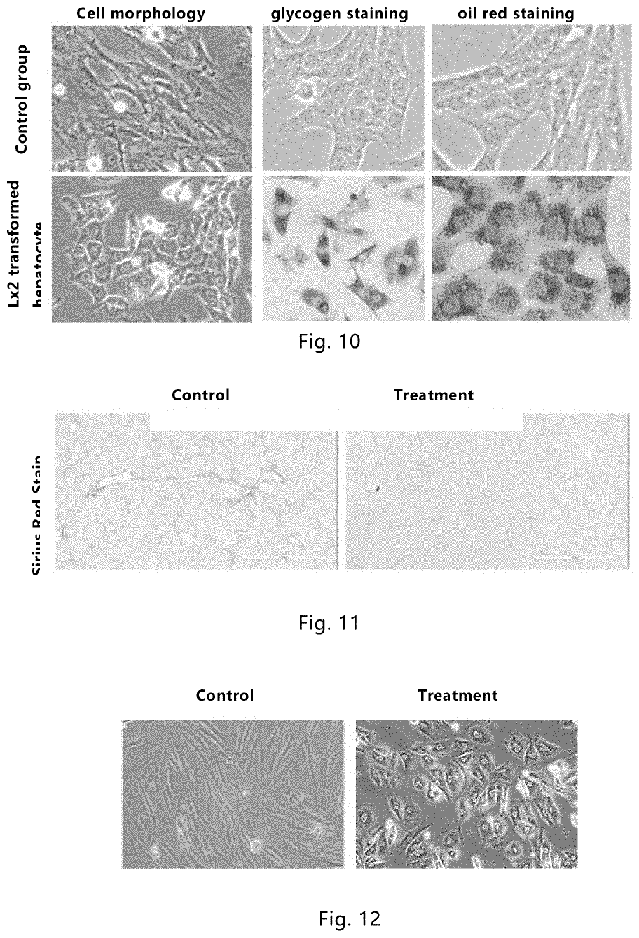

The method of inducing transdifferentiation of hepatic fibroblast Lx2 (hepatic stellate cell HSC, Lx2) into hepatocyte by the fibroblast to hepatocyte transdifferentiation Culture Medium 10 is the same as in Example 2. (1) Compare the morphology of human hepatic stellate cells Lx2 (control group) with the Lx2-transdifferentiated hepatocyte (Lx2-ciHep) (treatment group); (2) compare the glycogen staining of human hepatic stellate cells Lx2 (control group) with the Lx2-transdifferentiated hepatocyte (Lx2-ciHep) (treatment group) (the steps are the same as in Example 6); (3) Comparison of oil red staining of human hepatic stellate cells Lx2 (control group) with the Lx-2transdifferentiated hepatocyte (Lx2-ciHep) (treatment group) (according to the instructions of the kit) (the experimental results are shown in FIG. 10).

The morphology and function (Glycogen staining, oil red staining) of the Lx2-transdifferentiated hepatocyte (Lx2-ciHep) (treatment group) and human hepatic stellate cells Lx2 (control group) are compared. The results show that after transdifferentiation of human hepatic stellate cells Lx2 into hepatocyte-like cells, they have the morphological and functional characteristics of hepatocytes.

### Example 12: Treatment of Hepatic Fibrosis Mouse Model Through In Situ Transdifferentiation of Fibroblasts Induced by Oral Administered Small Molecule Reagent

Experiment Steps:

1. Construction of mice models with hepatic fibrosis: male C57/BL6 mice of 4 to 5 weeks old, injected intraperitoneally with 5% CCl₄ (olive oil solvent) at a dose of 5 μL/g body weight at 3 times/week, for 84 days. Hepatic fibrosis model should be successfully constructed at about 12 weeks. At the twelfth week of modeling, a mouse was dissected, and liver tissue were fixed and sliced for HE staining (conventional staining) and Sirius red staining (according to the instructions of the kit) to confirm the emergence of hepatic fibrosis.

2. Preparation of oral reagent for fibroblast transdifferentiation and treatment experiment: oral administration. The DMSO concentrated solution of fibroblast to hepatocyte transdifferentiation Composition 10 for was dissolved in 5% Captisol to prepare an oral reagent for fibroblast transdifferentiation (same concentration as the final concentration of the compound of Composition 10), give to treatment group (n=6) once per day; the same dose of DMSO was dissolved in 5% Captisol, and given orally to control group (n=6) once per day. Treatment period is 34 days long, or about 5 weeks, during which CCl4 continued to be injected into animals. After the experiment, the mice blood and liver are collected for subsequent analysis.

3. Liver tissues of mice from the treatment and the control group were fixed and sliced for Sirius Red staining (according to the operating instructions of the kit). The results show that the Sirius Red staining of the mice in the treatment group is significantly reduced, indicating that small molecule-induced fibroblast transdifferentiation can significantly alleviate or reverse liver fibrosis (see FIG. 11).

### Example 13 Experiment of Transdifferentiation of Human Lung Fibroblasts into Hepatocyte-Like Cells

The method for the induction of human lung fibroblast transdifferentiation into hepatocyte with fibroblast to hepatocyte Culture Medium 6 is the same as in Example 2. The experiment results are shown in FIG. 12. The results show that human lung fibroblasts gain the morphological characteristics of hepatocytes after transdifferentiation into hepatocyte-like cells.

### Example 14. After Transdifferentiating into Hepatocyte-Like Cells, the Human Lung Fibroblasts Express Hepatocyte-Related Genes

The method for the induction of human lung fibroblast transdifferentiation into hepatocyte with fibroblast to hepatocyte Culture Medium 6 is the same as in Example 2. The RNA of the cells in the control group and the transdifferentiated group was collected respectively, and RT-PCR was performed to detect the expression of hepatocyte-related genes.

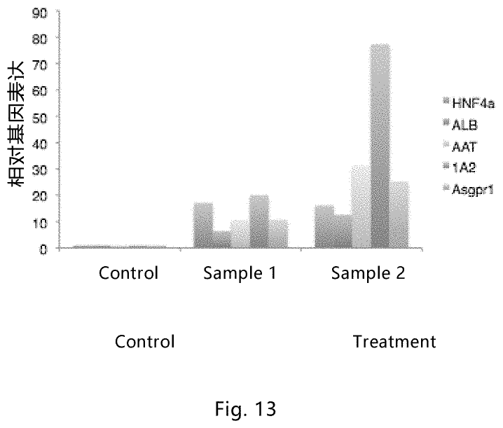

The results are shown in FIG. 13. Hepatocyte-related genes were highly expressed after lung fibroblasts were transdifferentiated into hepatocyte-like cells. This indicates that human lung fibroblasts have been transdifferentiated into hepatocytes.

In summary, the “method of inducing human fibroblasts to directly reprogram (transdifferentiate) into hepatocytes” of the present disclosure has the following characteristics:

1. Small molecules used are stable in nature, and can be easily manipulated through action time, dosage and ways of combination; their effects are also stable and consistent;

2. Fibroblast-transdifferentiated hepatocytes have the morphology and function of normal mature human hepatocyte. They have albumin production, urea synthesis, glycogen storage, P450 enzyme activity induction, and other hepatocyte-specific functions;

3. the fibroblasts in this method can be obtained from the patient and transdifferentiate into autologous hepatocytes, which have two major advantages: first of all, they can be applied clinically as they can minimize or avoid immune rejection risk in hepatocyte transplantation; secondly, they can be used for building crowd-representative hepatocyte bank for liver toxicity and efficacy screening of new drugs; therefore, the present disclosure can provide new cell sources for clinical and medical applications;

4. The present disclosure excludes introduction of exogenous genes (transcription factors), or making changes to cell's genetic sequence, and thus avoids new carcinogenic risks caused by the introduction of exogenous genes or genetic changes, therefore this method is safe, reliable, and suitable for clinical application;

5. This method uses small molecule combination to induce fibroblasts to reprogram directly into hepatocytes without going through the induced pluripotent stem cell (iPSC) stage, thus avoiding the risk of carcinogenesis by pluripotent stem cells;

6. small chemical molecules are stable in nature and can easily be developed into pharmaceutical drugs. This method can be easily applied to in situ cell transdifferentiation in vivo, and the composition can be used to develop or prepare drugs or prodrugs for treating human hepatic fibrosis (cirrhosis), pulmonary fibrosis, and fibrosis diseases of other organ or tissue.

7. The transdifferentiation method is universally applicable and repeatable;

8. The method is simple, easy to operate, and inexpensive; it can provide culture medium or reagents for fibroblasts transdifferentiation into hepatocyte for scientific research applications;

9. The process requires routine cell culture methods with short cycle, thus suitable for mass production and industrialization.

All documents mentioned in the present disclosure are incorporated by reference in this application, as if each document was individually incorporated by reference. In addition, it should be understood that after reading the above-mentioned teaching content of the present disclosure, those skilled in the art can make various changes or modifications to the present disclosure, and these equivalent forms also fall within the scope defined by the claims attached to this application.

1. A small molecule composition for chemically inducing the direct reprogramming of human fibroblasts into hepatocytes, characterized in that the composition comprises a GSK3β inhibitor, a TGFβ inhibitor and a G9aHMTase inhibitor; or the composition consists of GSK3β inhibitor, TGFβ inhibitor and G9aHMTase inhibitor.

2. The composition according to claim 1, wherein the composition comprises:
a GSK3β inhibitor: 5-80 parts by weight; or a final concentration thereof in a solution state: 0.1-20 uM;
a TGFβ inhibitor: 0.1-50 parts by weight; or the final concentration thereof in the solution state: 0.01-20 uM; and
a G9aHMTase inhibitor: 0.1-50 parts by weight; or the final concentration thereof in the solution state: 0.01-20 uM.

3. The composition according to claim 1, wherein the composition comprises:
a GSK3β inhibitor: 10-70 parts by weight; or a final concentration thereof in a solution state: 0.5-10 uM;
A TGFβ inhibitor: 0.5-40 parts by weight; or the final concentration thereof in the solution state: 0.05-10 uM; and
A G9aHMTase inhibitor: 0.5-40 parts by weight; or the final concentration thereof in the solution state: 0.05-10 uM.

4. The composition according to claim 1, wherein the GSK3β inhibitor includes a GSK3β signaling pathway inhibitor or a compound of the same class that have the same function or target the same induction site(s), as represented by CHIR-99021, BIO, IM-12, TWS119, 1-Azakenpaullone, CHIR-98014, Tideglusib, AR-A014418, LY2090314, SB216763, AZD1080, etc., or a functionally equivalent pharmaceutical preparation, analogue, isomer and/or salt, hydrate or precursor thereof, or a combination thereof.

5. The composition according to claim 1, wherein the TGFβ inhibitor includes a TGFβ signaling pathway inhibitor or a compound of the same class that have the same function or target the same induction site(s), as represented by SB431542, A83-01, SB525334, LY2109761, RepSox, SD-208, GW788388, SB505124, EW-7197, Galunisertib, etc., or a functionally equivalent pharmaceutical preparation, analogue, isomer and/or salt, hydrate or precursor thereof, or a combination thereof.

6. The composition according to claim 1, wherein the G9aHMTase inhibitor includes a G9aHMTase inhibitor or a compound of the same class that have the same function or target the same induction site(s), as represented by: BIX01294, UNC0638, A-366, UNC0631, BRD4770, UNC0224, UNC0646, UNC0642, etc.; or a functionally equivalent pharmaceutical preparation, analogue, isomer and/or salt, hydrate or precursor thereof, or a combination thereof.

7. The composition according to claim 1, wherein the GSK3β inhibitor is GSK3β inhibitor CHIR-99021, BIO, CHIR-98014 or TWS119; and the TGFβ inhibitor is TGFβ inhibitor SB431542, A83-01 or LY2109761; or the G9aHMTase inhibitor is G9aHMTase inhibitor BIX01294 or UNC0638.

8. The composition according to claim 1, wherein in the composition, the GSK3β inhibitor (such as GSK3β inhibitor CHIR99021), the TGFβ inhibitor (such as the TGFβ inhibitor SB431542 or/and A83-01), G9aHMTase inhibitor (such as G9aHMTase inhibitor BIX01294), in parts by weight: (5-80):(0.1-50):(0.1-50); or in a solution state at a molar concentration ratio: (0.1-20):(0.01-20):(0.01-20).

9. The composition according to claim 8, characterized in that in the composition, the GSK3β inhibitor (such as the GSK3β inhibitor CHIR99021), the TGFβ inhibitor (such as the TGFβ inhibitor SB431542 or/and A83-01), and the G9aHMTase Inhibitors (such as G9aHMTase inhibitor BIX01294) are present in parts by weight: (10-70):(0.5-40):(0.5-40); or in a solution state at a molar concentration ratio: (0.5-10):(0.05-10):(0.05-10).

10. The composition according to claim 1, wherein the composition is a pharmaceutical composition for treating hepatic fibrosis (cirrhosis), pulmonary fibrosis, and fibrosis diseases of other organ or tissue of human body, further comprising a pharmaceutically acceptable carrier or excipient, the carrier or excipient comprising one or more selected from the group consisting of water, saline, phosphate buffer or other aqueous solvents; DMSO, glycerol and ethanol or other organic solvents; microspheres, liposomes, microemulsions or macromolecular surfactants; colloidal drug-loading systems or macromolecular drug-loading systems; preservatives, antioxidants, flavoring agents, fragrances, co-solvents, emulsifiers, pH buffer substances, adhesives, fillers, lubricants or other pharmaceutical excipients; or
the pharmaceutical dosage forms of the composition comprise: solid dosage forms, including powders, pulvis, tablets, pills, capsules, sustained release agents, controlled release agents, or other solid dosage forms; liquid dosage forms, including injections, infusions, suspensions, or other liquid dosage forms; gas dosage forms; or semi-solid dosage forms.

11. A method for developing or preparing of drug or pre-drug for treating human hepatic fibrosis (cirrhosis), pulmonary fibrosis, and fibrosis diseases of other organ or tissue of the human body wherein said method comprises mixing the small molecule composition for chemically inducing the direct reprogramming of human fibroblasts into hepatocytes with a pharmaceutically acceptable carrier or excipient.

12. A method for inducing fibroblasts to directly reprogram into hepatocytes, characterized in that the method comprises: inducing human fibroblasts to directly reprogram into hepatocytes by using the composition of claim 1.

13. A kit or test kit for inducing human fibroblasts to directly reprogram into hepatocytes, characterized in that the kit or test kit comprises: the composition according to claim 1; or a drug or prodrug for treating human hepatic fibrosis (cirrhosis), pulmonary fibrosis and fibrosis diseases of other organ or tissue developed based on the composition; or a cell transdifferentiation reagent or medium prepared based on the composition.

14. The composition according to claim 1, wherein the human fibroblasts comprise, but are not limited to, fibroblasts from different tissues or organs of the human body, such as skin fibroblasts, hepatic fibroblasts (hepatic stellate cells), lung fibroblasts, kidney fibroblasts, pancreatic fibroblasts, and fibroblasts from other tissues or organs of the human body.

15. A method for preparing media or reagents that induce direct reprogramming of human fibroblasts into hepatocytes, wherein said method comprises mixing the small molecule composition for chemically inducing the direct reprogramming of human fibroblasts into hepatocytes with a a basal cell culture medium; or with an aqueous solvent or an organic solvent.

16. The method according to claim 12, wherein the human fibroblasts comprise, but are not limited to, fibroblasts from different tissues or organs of the human body, such as skin fibroblasts, hepatic fibroblasts (hepatic stellate cells), lung fibroblasts, kidney fibroblasts, pancreatic fibroblasts, and fibroblasts from other tissues or organs of the human body.

17. The kit or test kit according to 13, wherein the human fibroblasts comprise, but are not limited to, fibroblasts from different tissues or organs of the human body, such as skin fibroblasts, hepatic fibroblasts (hepatic stellate cells), lung fibroblasts, kidney fibroblasts, pancreatic fibroblasts, and fibroblasts from other tissues or organs of the human body.
